(19) United States
(12) Patent Application Publication
(10) Pub. No.: US 2004/0219668 A1
(43) Pub. Date: Nov. 4, 2004

(54) Method and device for the in vitro cultivation of cells

(76) Inventors: Heribert Frei, Winterthur (CH); Pierre Mainil-Varlet, Bern (CH); Werner Muller, Wiesenbangen (DE)

Correspondence Address: RANKIN, HILL, PORTER & CLARK LLP, 4080 ERIE STREET, WILLOUGHBY, OH, 44094-7836, US

(21) Appl. No.: 10/488,018

(22) PCT Filed: Aug. 29, 2002
(86) PCT No.: PCT/CH02/00471

(30) Foreign Application Priority Data
Aug. 30, 2001 (CH) 1619/01

(51) Int. Cl. C12N 5/08; C12N 5/02

(52) U.S. Cl. 435/366; 435/404

(57) ABSTRACT

A method for in vitro cultivation of cells, which grow on a culture surface, wherein the cells are sown on culture surfaces (1) and are cultivated in a culture medium, whereby the culture surface is continually or periodically expanded, without being removed from the culture medium. To expand the culture surface, the cells are not detached from the culture surface and the culture surface between the cells is expanded. However, at least part of the cells can be detached and the culture surface can be expanded by the flooding of additional culture surface areas. The culture surface of an example device for cell cultivation consists of one side of an expanding membrane (6), which is expanded by modifying the pressure on the other side. The cell cultivation can be carried out without manual passaging. The cells are subjected to less stress than in known cell cultivation methods and the method can be automated more easily.

The invention lies in the field of in vitro cell cultures and relates to a method and to a device according to the preambles of the corresponding, independent patent claims. Method and device according to the invention serve for in vitro proliferation of cells which adhere to a culture surface.

From a plurality of publications e.g. from Fuss et al. “Characteristics of human chondrocytes, osteoblasts and fibroblasts seeded onto a Type I/III collagen sponge under different culture conditions”, Anat. Anz. 182:303-310 (2000); Chan et al. “A new technique to resurface wounds with composite biocompatible epidermal graft and artificial skin”, J. Trauma 50:358-362 (2001); Roth et al. “Nonviral transfer of the gene encoding coagulation factor VIII in patients with severe haemophilia A,” N. Engl. J. Med. 344:1735-1742 (2001), it is known to remove cells from a patient's tissue (autologous cells) and, after proliferation in culture, to transfer the cells back into the body of the patient (cell autotransplantation). The main advantages of cell autotransplantation compared with organ transplantation are the following: no risk of infection with diseases since own cells are used and no limitation due to the limited number of organ donors and to conditions regarding histocompatibility between donor and receiver. Furthermore, it is easier to plan operation schedules.

For the autotransplantation of cells, a small tissue sample is taken from the body of the patient in a first, small operation. Vital cells are then isolated from the tissue sample and proliferated in vitro. In a second operation, a suspension of the proliferated cells is implanted back into the patient. In vivo, the implanted cells form a tissue equivalent, which assumes the function of the original tissue. There are also known methods for growing tissue equivalents in vitro from the proliferated cells (tissue engineering). The engineered tissue, which constitutes a more or less mature precursor of a functional tissue, is then implanted in the patient.

According to the state of the art, in vitro proliferation of tissue cells is carried out without exception by highly qualified personnel and essentially manually, if the cells cannot be cultured in suspension but must adhere to a culture surface. Once the cells have proliferated in such culture, they are detached from the culture surface with the aid of trypsin or other enzymes. They are then separated from the enzyme, re-suspended in a medium containing no enzyme and re-seeded with a lower number of cells per culture surface unit. The lower cell density with which the cells are re-seeded permits further cell proliferation. This succession of working steps is known as “cell passaging ”. Often, the necessary periodic exchange of the culture medium is carried out manually also. There are a plurality of disadvantages inherent in the named known cell culture methods. Particularly disadvantageous is the treatment of the cells with trypsin or generally speaking with enzymes because this treatment damages the cells usually irreversibly and to an unknown extent. Furthermore, all method steps which are carried out manually constitute high personnel cost and necessitate an extensive quality control. In addition, all manually executed method steps constitute an increased infection risk for the cell cultures and, in the case of a clinical application, also to the patient. Moreover with all manual work regarding human cells there is a risk of infection to the laboratory personnel.

There are known bioreactors suitable for culturing cells which adhere to a culture surface. Such bioreactors comprise a two-dimensional culture surface (e. g. described in WO-96/40860) or a three-dimensional matrix (e.g. described in FR-2768783-A1 or in WO-01/14517-A1) to which the cells adhere. The cells are seeded on the two-dimensional culture surface, are cultured for proliferation and are then harvested for autotransplanation. The three-dimensional matrix in which the cells are likewise seeded and cultured are usually used directly as so-called ex vivo organ parts. The bioreactors are equipped with control systems for maintaining the culture medium, the gas exchange and other culture parameters within predetermined limits.

Costs with regard to personnel as well as to quality control can be saved when using instead of the fully manual procedure the above described bioreactors. The decisive disadvantage of these bioreactors for clinical application is the fact that proliferation of the cells is limited by the available culture surface. The same applies to bioreactors according to EP-0725134 and WO-0066706 which comprise flexible walls and culture surfaces, and to bioreactors according to WO-00/12676 which comprise elastic walls. If the limited number of cells resulting from cell proliferation in the named bioreactors is not high enough and therefore the cells need to be further proliferated, cell passaging becomes again necessary. The bioreactors can therefore not alleviate the main disadvantage to the biology of the cells, since, on being passaged, the cells are contacted with trypsin and/or other enzymes and thereby suffer irreversible and uncontrollable damage.

For passaging cells which adhere to a culture surface, usually the culture medium is separated from the cell culture and is replaced by the enzyme solution. Through the effect of the enzyme, the cells are detached from the culture surface and, if so applicable, they are also separated from neighbouring cells, such that enzyme treatment results in a suspension of individual cells. The suspended cells are then washed and re-seeded with a lower cell density on a new culture surface which is usually selected to be larger than the preceding culture surface, and the cells are further proliferated in culture medium.

It is known that most cell types which adhere to a culture surface proliferate optimally when present on the culture surface in a number per surface unit, which number varies within a cell density range determined in particular by the cell type. For a mutual, favorable influencing, the cells should not be too distanced from one another, and for an unhindered proliferation they should not be too close to one another. In cultures with cell densities outside the mentioned cell density range, cells are lost, cell proliferation is reduced and/or cell differentiation is changed in an accelerated manner. For these reasons, cells in culture, in particular cells having a low cell density tolerance need to be passaged relatively often.

As mentioned further above, due to the enzyme treatment, passaging is a great biological burden to the cells. In particular, irreversible changes of components of the cell surface occurring on passaging may influence the function and differentiation of the cultured cells.

From the above described knowledge of cell proliferation in culture it follows that improving cell culture should regard the passaging step, i.e. it should reduce the burden that passaging puts on the cells, in such a manner that the cells can be passaged more often, or it should change known culture methods such that passaging in the conventional sense is no longer necessary. The object of the present invention is therefore, to create a method and a device for proliferating cells in culture, in which the cells adhere to a culture surface, wherein method and device are to allow high cell proliferation, in a manner such that compared to known cell culture methods comprising manual passaging, the overall burden to the cells due to passaging is lower, and despite this, the cell density on the culture surface can be kept within a narrower range. Furthermore, method and device according to the invention are to constitute a lower risk of contamination compared to known methods and devices, and are to be suitable in particular for culturing epithelial cells and connective tissue cell types.

This object is achieved by the method and the device as defined in the patent claims.

According to the invention, the culture surface which is made available to the cells to be proliferated is enlarged during uninterrupted cell culture, wherein the increase in culture surface size, just as with passaging, is adapted to the cell number which is growing due to the cell proliferation. For the culture surface enlargement, the cells which adhere to the culture surface are not removed from the culture medium. The culture surface is enlarged between the cells adhering to it in all its regions and in the smallest of steps, so that the reduction of the cell distances caused by cell proliferation is compensated so to speak continuously and the cell density remains essentially constant or is maintained within in a very narrow range. Alternately, a part of the cells are detached from the culture surface and are brought into suspension continuously or in small time intervals and further culture surface regions not yet colonized are made available to the suspended cells. The same can also be achieved by detaching all cells from the culture surface but without the necessity of replacing the culture medium by an enzyme solution (for example by way of mechanical means), and by simultaneously making available to the cells, further, not yet colonized culture surface regions.

According to the invention, the cells are either not detached from the culture surface to which they adhere, or this is carried out with more gentle measures, such that even with relatively frequent detachment, the burden to the cells remains within tolerable limits. This allows to enlarge the culture surface to which the cells adhere in smaller steps than with known methods or it allows to enlarge it in an essentially continuous manner, such allowing cell proliferation with a less varying cell density than is possible when using known passaging methods. It is found, that in cell cultures operated according to the invention, not only more cells survive than in known culture methods, but also even on high cell proliferation, cell differentiation is changed less than in known culture methods. The well known fact that cell function and differentiation and further cell properties depend on the cell density during cell culture, explains that by using method and device according to the invention allows to produce cells having predefined characteristics depending on the chosen cell density. Since method and device according to the invention allow cell proliferation with a cell density that varies less over time than in known cell culture methods, the cell characteristics within one cell culture will scatter less. The low scatter of the cell characteristics is a significant experimental advantage for many applications, or it is even an experimental precondition for the results of experiments to achieve significance, or to achieve any results which can be interpreted against the experimental background scatter.

The device according to the invention comprises a culture surface to be positioned in a culture medium and being suitable for cell adhesion. The device further comprises means for enlarging the culture surface while it remains positioned in the culture medium. The enlarging means are controlled in a manner such that the culture surface enlargement, just as with passaging, is adapted to the growing of the cell number which is due to proliferation. The device further comprises, in the same way as known bioreactors, means for periodical or continuous renewal of the culture medium. If applicable, the device further comprises means for detaching at least part of the cells from the culture surface.

The cells cultured according to the invention are suitable for applications in cell biology or in molecular biology, for autotransplantation and for other applications.

The device according to the invention may be combined with technical means for on-line monitoring of the cell proliferation, for example via measurement of scattered light and/or indirectly via measurement of culture parameters (e.g. pH-value in the culture medium), in order to control cell proliferation to be maintained within predefined limits, or for exchanging the culture medium in a predefined manner. This allows to adapt devices according to the invention to demands of the most varied of application fields in a very flexible manner.

The devices according to the invention may be realized to be completely or partly disposable or to represent reusable apparatus. Such they are capable of being used in very different application fields such as cell culture research, industry, diagnostics and clinically. This leads to unexpectedly simple, safe and inexpensive solutions for cell and tissue culture in various application fields.

The devices according to the invention also open up the possibility of not only continuously or stepwise enlarging the culture surface during cell proliferation, but also of reducing it. This opens the way to completely new culture conditions. For example, it becomes possible to simulate in vitro phases of organ or tissue development of multi-cell organisms, in which phases the cell density changes. The cell-to-cell contacts and the mutual influencing of the cells by way of autocrine factors can be fully exploited for cell culture and tissue engineering by way of controlling the cell density or the distances between cells respectively.

The culture surfaces of the device according to the invention may be pre-treated in per se known manner for optimal cell attachment and/or for a desired cell or tissue differentiation. The pre-treatment may be effected, for example, by glow discharge or plasma, by coating with molecules of a specific extra-cellular matrix or with mixtures of components of the extra-cellular matrix, by biological build-up of layers of the extra-cellular matrix through feeder cells, by chemical modification of the charge density or by bonding functional groups and/or signal molecules adapted to cell receptors, etc.

The invention is hereinafter described by way of exemplary embodiments of the device according to the invention, but is not limited to the shown embodiments. Herein:

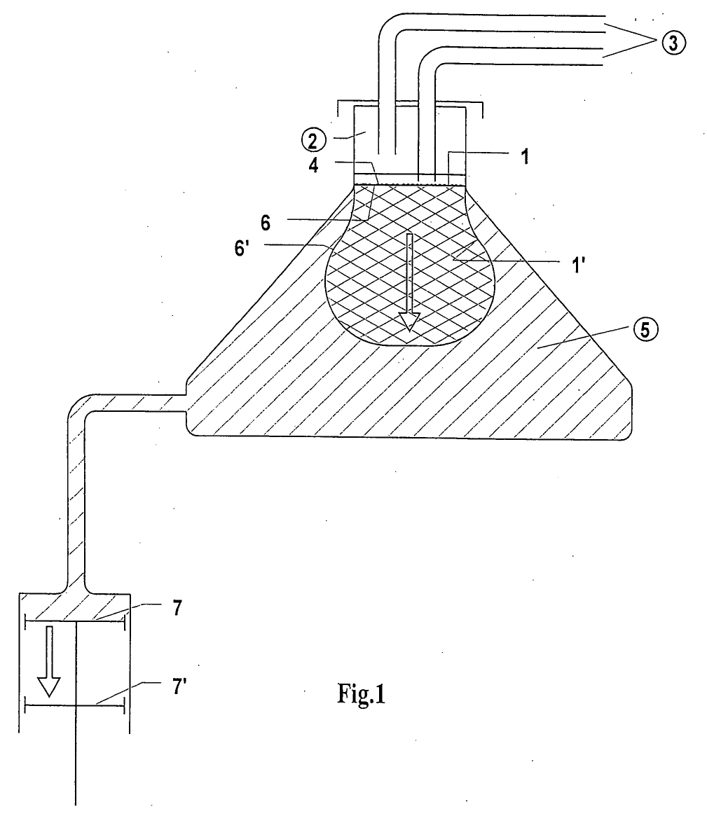

FIG. 1 is a section through a first, exemplary embodiment of the device according to the invention, the device comprising a culture surface on an expandable membrane;

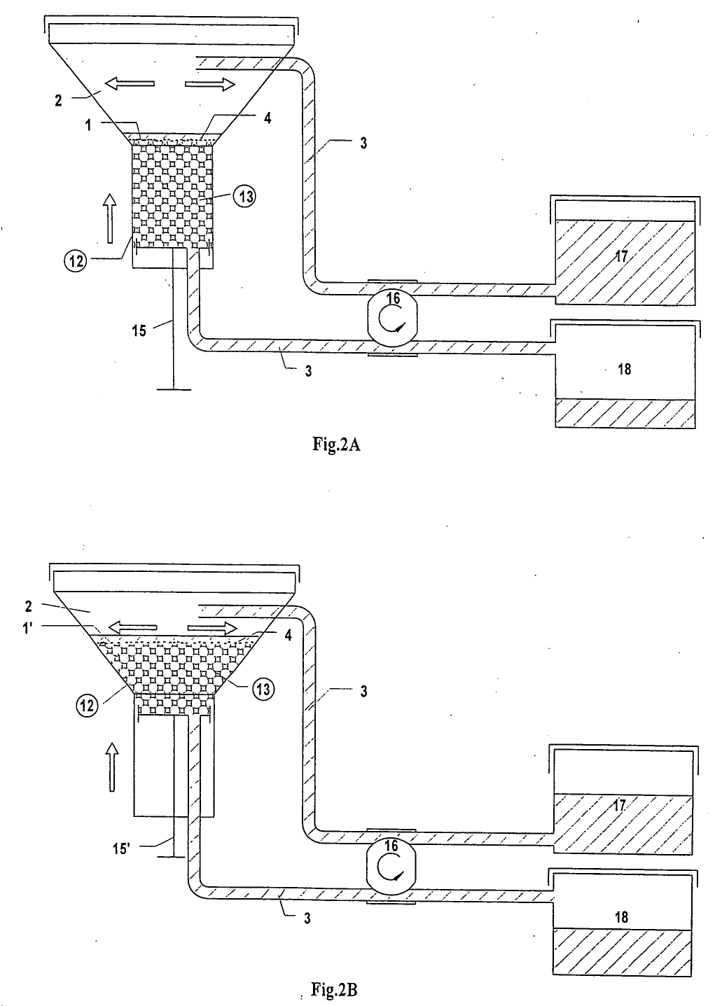

FIGS. 2A and 2B are sections through a further exemplary embodiment of the device according to the invention, the device comprising a culture surface formed by a large number of small particles;

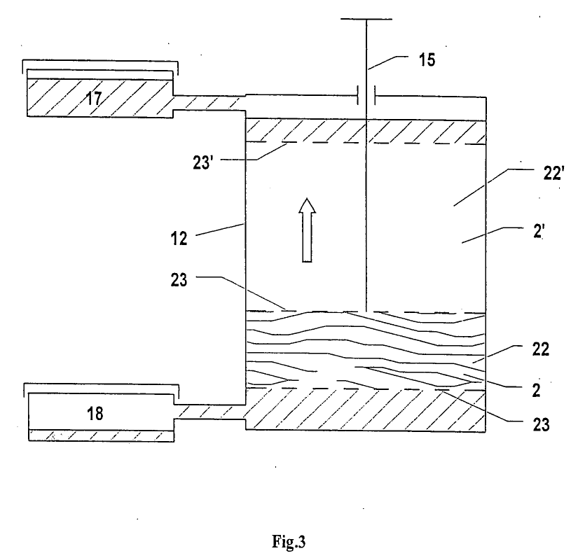

FIG. 3 is a section through a further exemplary embodiment of the device according to the invention, the device comprising a culture surface which is formed by the inner surface of a compressible, open-pored body;

FIG. 4 is a section through a further exemplary embodiment of the device according to the invention, the device comprising means for producing a current in the culture medium, through which current a part of the cells are detached from the culture surface, and means for flooding with culture medium further culture surface regions for being colonized by the detached cells;

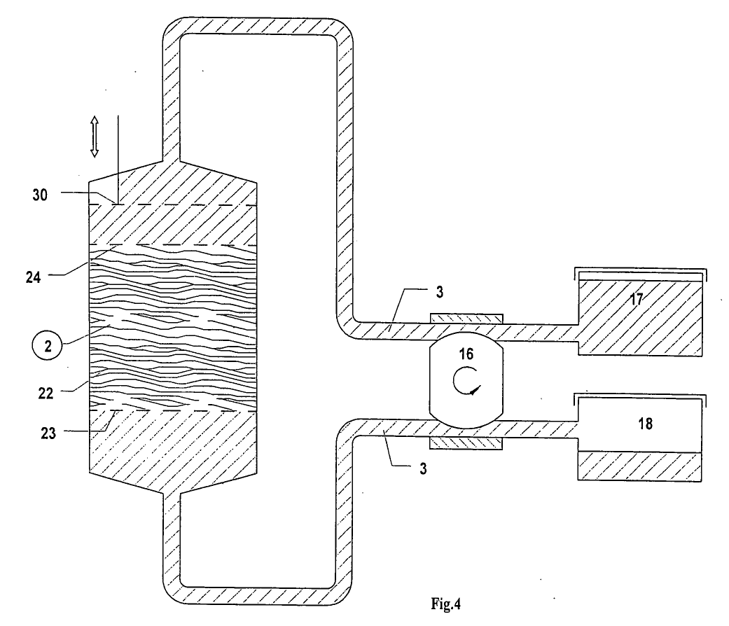

FIG. 5 is a section through a further exemplary embodiment of the device according to the invention, the device comprising culture surfaces on conduits comprising semi-permeable walls for cell detachment with the aid of enzymes, and means for flooding with culture medium further such conduits for being colonized by detached cells;

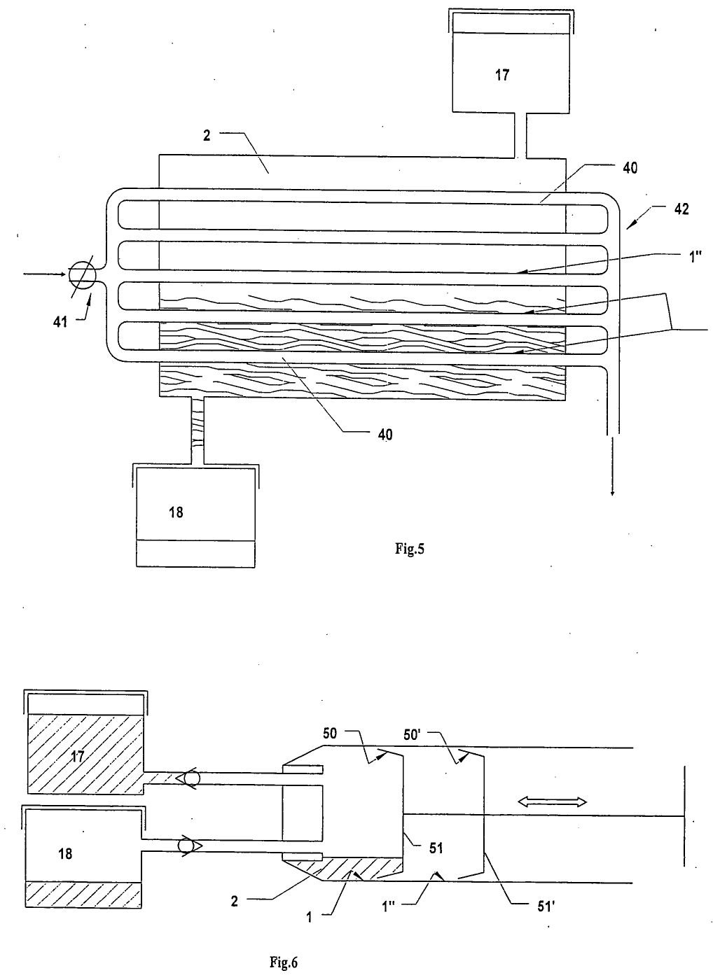

FIG. 6 is a section through a further, exemplary embodiment of the device according to the invention, the device comprising means for mechanically detaching the cells from the culture surface and means for flooding with culture medium further culture surfaces to be colonized by detached cells;

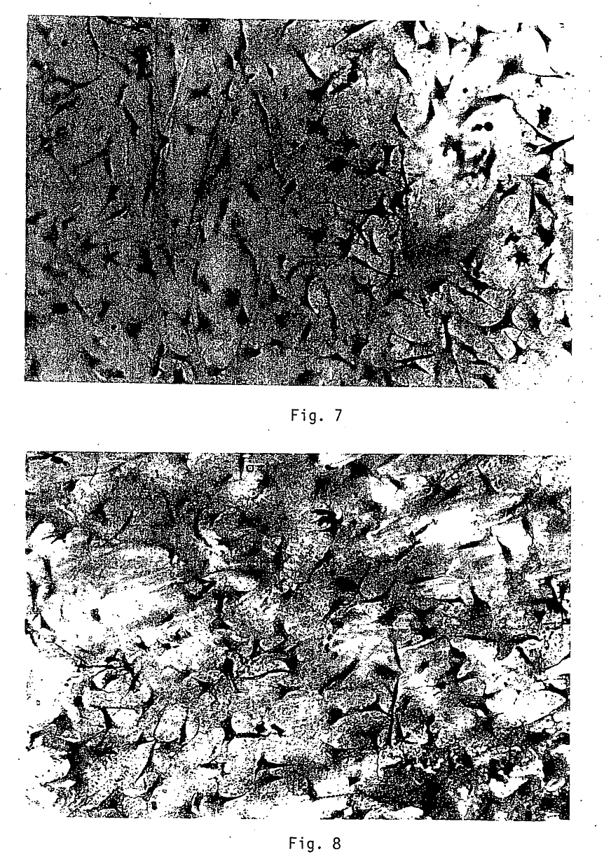

FIG. 7 is a micro-photographic picture of cells proliferated according to Example 1 on a non expanding membrane (colouring: Mayer's hernalum);

FIG. 8 is a micro-photographic picture of cells proliferated according to Example 1 on a membrane being expanded during cell proliferation (colouring: Mayer's hernalum).

FIG. 1 shows an exemplary embodiment of the device according to the invention, the device comprising a culture surface 1 constituted by the surface of an expandable membrane 6. The culture space 2 is situated on one side of the membrane 6 and is equipped with suitable supply and removal conduits 3 for the renewal of the culture medium. A further space 5 is situated on the other membrane side, is filled with gas or fluid and is equipped e.g. with a plunger 7 for reducing the gas or fluid pressure.

The membrane 6 is fastened in an essentially unexpanded condition between the culture space 2 and the further space 5. The cells 5 are seeded on the membrane surface (culture surface 1) which faces the culture space 2 and are covered with culture medium. The medium is renewed continuously or periodically during the cell culturing in per se known manner. During cell culturing, the membrane 6 is expanded continuously or periodically (stepwise) by continuously or periodically reducing the pressure in the further space 5. Through pressure reduction, the membrane 6 is deformed to become more and more concave and the culture surface 1 is therewith enlarged.

Convex deformation and enlargement of the culture surface 1 may be realized in the same manner by way of increasing the pressure in the further space 5.

The membrane 6, the culture surface 1 and the plunger 7 of the device according to FIG. 1 are in each case shown in an initial position in which they are indicated with the mentioned reference numerals, and at a later stage of the cell culture indicated with the same reference numerals comprising an apostrophe (1′, 6′, 7′).

The membrane 6 of the device according to FIG. 1 is for example a dental membrane (e.g. dental membrane available under the trade names of “non-latex Dental Dam” or “Flexi Dam non latex” by ROEKO, D-89122 Langenau, Germany), or any other membrane on which cells may be cultured and proliferated, and which is preferably expandable by more than fourfold to tenfold. The material and structure of the culture surface is to permit cells to adhere to and proliferate on this surface. For this reason, as the case may be, the membrane needs to be modified or coated using per se known methods, for example coating with fibronectine, collagen, gelatine, etc.

Gassing of the culture space may be effected via the further space 5 by using a gas-permeable membrane 6 and a liquid in the further space 5.

The culture space 2 of the device according to FIG. 1 may be closed and operated with per se known systems. For example, the culture medium is exchanged without opening the culture space 2 by using supply and discharge conduits 3. Furthermore measuring systems for recording and controlling culture parameters may be integrated in the device. The exemplary embodiment of the device as shown in FIG. 1 may be designed to have a more suitable form with regard to technology.

FIGS. 2A and 2B show a further, exemplary embodiment of the device according to the invention in a stage at the start of cell culture (FIG. 2A) and during cell culture (FIG. 2B). The culture surface 1 in this embodiment is formed by the upper surface of a volume 13 containing a large number of small particles and being arranged in a container 12, whose cross section increases in an upward direction. A suitable means (e.g. pusher 15) is provided in container 12 for pushing the particle volume 13 upwards such enlarging its upper surface (culture surface 1) by pushing further particles between the particles constituting this upper surface. Supply and removal conduits 3, a pump 16, a supply container 17 and a waste container 18 are provided for renewing the culture medium.

In FIG. 2B the device is shown in a condition in which the culture surface 1′ is enlarged with respect to the initial condition (FIG. 2A). Pusher 15′ is in a raised position.

The particles used in the device according to FIGS. 2A and 2B consist for example of glass, ceramics, plastics (e.g. polyurethane), etc. The particles may for example be spherical, rod-shaped, etc and typically have a size not exceeding 5 mm in any direction.

FIG. 3 shows a further, exemplary embodiment of the device according to the invention, the device comprising a compressible, open-pored body 22, for example a sponge, whose inner surface constitutes the culture surface. This inner surface is small in an initial state in which body 22 is compressed by pusher 15 (only a few of the pores are open). During cell culturing the inner body surface is enlarged by relaxation of the body through which the number of open pores is increased and their lumen is enlarged.

For compression and relaxation, the compressible, open-pored body 22 (and 22′) is for example arranged between two sieve-like, mutually displaceable carrier plates 23 (and 23′) through which the culture medium flows in an unhindered manner. The exchange of the culture medium is effected from the supply vessel 17 into the waste container 18 via the culture space 2 (and 2′).

FIG. 4 shows a further, exemplary embodiment of the device according to the invention, which embodiment is based on the fact that cells partly detach from the culture surface and assume a spherical shape when they prepare for cell division. In this cell state, adhesion to the culture surface is weakened and the cell surface engaged by shear forces increased so that cells being in a division phase can be tom from the culture surface using relatively small shear forces, in particular shear forces which are too weak for detaching cells not being in the cell division phase. The detached cells are then seeded onto culture surfaces which are made newly available for cell culturing.

The named phenomenon is exploited for detaching only a part of the cells from the culture surface, such giving to the cells remaining attached more space for further proliferation and to the detached cells the opporunity to attach to new culture surface regions, wherein neither the detached nor the remaining cells are burdened by enzyme treatment as no enzyme solution is used for cell detachment. The shear forces required for detaching the cells are created by culture medium currents which at the same time serve for suspending and distributing the detached cells for being able to colonize the culture surface regions which are made newly available.

The device according to the invention shown in FIG. 4 comprises for example a cylindrical culture space 2 in which again a compressible, open-pored body 22 is arranged for being compressed and relaxed between a carrier plate 23 and a plunger 30 both being permeable to the culture medium. The higher the plunger 30 is positioned in the culture space 2, the more relaxed is the compressible body 22, which means the larger is its inner culture surface.

The idle position of the plunger 30 being displaced upwards during cell culture is selected such that the cell density in the compressible body 22 always lies in a predefined range.

The culture medium current or surge required for partial cell detachment is produced by shock-like movements of the plunger 30 by which the momentary compression of the compressible body 22 is increased lightly for a short time. Such shock movements are repeated periodically, the time between shocks being at least as long as the time needed by a cultured cell for a complete cell division cycle, i.e. from the prophase to completion of the telophase.

For achieving the culture medium surge necessary for cell detachment in the compressible body 22, its inner structure may for example be formed as a capillary filter having a main direction in the direction of the culture medium current. The efficiency of the plunger 30 may further be enhanced for example by way of valve mechanisms arranged therein, the valve mechanisms closing when the current is strong (surge for cell detachment), and in contrast remaining open with normal current (exchange of the culture medium).

The device according to the invention as shown in FIG. 4 may also be designed as follows. A non-compressible, open-pored body 22 is arranged in the cylindrical culture space 2 between two carrier plates 23 and 24 being permeable to the culture medium. The shear force necessary for detachment of cells in division is produced by the pump 16 for example via the large-lumen supply and discharge conduit 3 or by the plunger 30.

FIG. 5 shows a further exemplary embodiment of the device according to the invention, the device comprising a culture surface 1 being constituted by a plurality of conduits 40 which run through the culture space 2 at various levels and whose walls are permeable to an aqueous enzyme solution. The conduits 40 are supplied individually and selectively with media with or without enzymes to flow from an entrance side 41 to an exit side 42. For detaching the cells from a specific conduits 40, medium containing enzyme is flown through the conduit and reaches the basal side of the cells and also cell-to-cell connections through the conduit wall, wherein contact with enzyme solution of the cell side facing the culture space 2, i.e. not in direct contact with the conduit wall remains minimal. For enhancing the named effect, enzyme inhibitors may be added to the culture medium in the space 2, e.g. an inhibitor specialized for inhibiting the one enzyme used and/or a serum. By being detached from a conduit 40, cells 4 are released into an essentially enzyme-free medium in which they are suspended by an increased current to be re-seeded distributed on several conduits 40, which are made available to them. The culture medium current is then stopped until the cells have settled and adhered on the culture surface 1.

For starting cell culture, cells are seeded on the conduits 40 of the lowermost level and only this conduit level is flooded with culture medium. When the cells on these conduits have reached a desired cell density, enzyme solution is flown temporarily through the conduits to pass through the conduit wall and to meet the cells in order to at least partly detach them by the enzyme effect. The flow of the enzyme solution is stopped immediately after cell detachment, by e.g. flowing culture medium through the conduits instead of the enzyme solution. The culture medium in the space 2 is circulated more rapidly to achieve more current for suspending the detached cells and for deactivating/neutralising and removing enzymes which as the case may be have got into the culture medium. The culture surface (1 and 1″) is enlarged for accommodating the detached cells by raising the culture medium level in the space 2 such that a second or further conduit level (additional culture surface regions 1″) is flooded. Circulation of the culture medium is then stopped until all cells are again adhered on the flooded conduits 40.

With the device according to FIG. 5 an enzyme solution (e.g. a trypsin solution) is used for detaching the cells. However, as this solution essentially only comes into contact with the basal side of the cells (the cell side adhering to the culture surface) while other cell sides are still positioned in the culture medium, the burden to the cells by the enzyme is significantly lower than on manual passaging.

FIG. 6 shows a further, exemplary embodiment of the device according to the invention, the device comprising means for mechanical detachment of the cells from the culture surface and means for enlarging the culture surface.

The culture surface 1 has the shape of a hollow cylinder and the cells are detached with a suitably shaped blade 50 which is fastened on the end side of a plunger 51, the plunger being axially displaceable in the hollow cylinder. A brush or a rubber scraper (rubber policeman) may be provided for detaching the cells instead of the blade 50.

For cell detachment, the plunger 51 is moved into the culture space 2. For enlarging the culture surface 1 (addition of further culture surface regions 1″) it is retracted more and more from the hollow cylinder (positions 50′ and 51′).

The invention is hereinafter described by way of the example of chondrozyte culturing, but it is not limited to this cell type.

### EXAMPLE 1

Example 1 relates to a cell culture in a device as illustrated by FIG. 1.

An expandable membrane from the dental field was used (Hygienic® NON-LATEX DENTAL DAM, Coltene/Whaledant Inc., USA). Further used elements were standard materials from a cell culture laboratory. The used device was prepared using a disposable plastic syringe. The membrane was washed three times for 10 min. with sterile phosphate-buffered saline solution. Then it was positioned in 70% ethanol three times for 10 min each time and then dried in a sterile workbench. The device was assembled in the sterile workbench using sterile gloves. The membrane was fastened on the sectioned cylinder of the plastic syringe with the aid of a piece of silicone tubing.

The assembled device was treated twice for 15 min. with 70% ethanol, then twice for 10 min. with phosphate-buffered saline solution and before seeding the cells on the membrane it was treated twice for 10 minutes with culture medium. Before seeding the cells the space between the syringe plunger and the expandable membrane was filled with culture medium using a syringe with an injection needle. Care was taken for the space to be free of gas and the membrane to form a planar surface.

D-MEM/F12=1:1 (Life Technologies, Basel, Switzerland) with L-glutamate and 10% foetal calf serum (HyClone, Utah, USA) was used as a culture medium wherein the buffer concentration was increased to 35 mM by adding HEPES (Life Technologies, Basel, Switzerland), in order to be able to carry out the cell culture without CO₂ gassing. The cells were detached from the culture surface with trypsin (Life Technologies, Basel, Switzerland).

Chondrocytes from knee joints of 6-month-old calves were used as test cells. The chondrocytes were isolated from the joint cartilage with pronase (2.5 mg/ml; Roche, Switzerland) and subsequently with collagenase (2.5 mg/ml; Roche, Switzerland) and cultivated in culture medium D-MEM/F12 with 15 mM HEPES and 10% foetal calf serum in plastic culture bottles with 5% CO₂ gassing. The cells in each case on reaching confluence were detached from the culture surface with trypsin and were re-seeded into new culture bottles. The cells were passaged three times in this manner before they were used in the following experiment.

The chondrocytes were seeded with a density of 10,000 cells/cm² on 1.8 cm² of the unexpanded culture surface. D-MEM/F12 with 35 mM HEPES and 10% foetal calf serum was used as a culture medium. The apparatus was protected from infection with a small petri-dish lid. In order to prevent an undesired displacement of the plunger, the plunger rod was secured with an artery clamp. For culturing, the apparatus was placed in a heated cupboard at 37° C. for culturing.

The cells were seeded manually and the culture medium was changed manually. During chondrocyte culture, the plunger of the 10 ml syringe was pulled downwards each day by 0.5 ml, whereby the membrane surface was stepwise enlarged. In each case 0.5 ml of culture medium was added to the culture space for supplementing the volume. In control devices 0.5 ml of culture medium was likewise added, but the plunger was left in its initial position, i.e. the membrane was not expanded.

After 10 days the cultures were washed with phosphate-buffered salt solution. The cells were subsequently harvested with trypsine. Of the harvested cells, a portion was cultivated further under the same conditions as before the experiment, and were evaluated qualitatively with regard to morphology for the next four days using an inverted microscope. Another portion of the harvested cells was dyed with trypan blue and the number of living and dead cells was counted in a haemocytometer. Further cultures were fixed in situ after washing using 4% formaldehyde solution and were then dyed with Mayer's Hamalum. The expanded membrane was then carefully removed from the apparatus. On removal from the apparatus the membrane did not return to its original size but remained partly expanded and therefore non planar. For this reason it had to be partly cut open in order to be fastened on an object carrier and to be covered with a cover glass. The cells adhering to the membrane were then examined and photographed in an epimicroscope.

Qualitative comparison of the cell morphology in the cultures before and after the experiment, as well as after completion of the control culture (on the unexpanded membrane; FIG. 7) and of the experimental culture (expanded membranes; FIG. 8) resulted in no evident differences with respect to the morphology of the cells. No dead cells were observed when determining the cell number. The total number of living cells after 10 days is indicated in Table 1.

TABLE 1

| | seeding | harvest | |
|---|---|---|---|
| | total cells | total cells | cultivation |
| | a × | b × | factor |
| Sample | 10⁵ | 10⁵ | v = b/a |
| control | 0.18 | 0.48 | 2.67 |
| trial | 0.18 | 2.40 | 13.33 |

The results show that the cells proliferated on the expanded membrane. The morphology of the cells on the expanded membrane (experiment) was comparable to the morphology of the cells on the unexpanded membrane (control). The number of cells which were harvested from the expanded membrane was roughly five times larger than the number of cells harvested from the unexpanded membrane. On further culturing of the cells, no difference with respect to cell morphology and cell density was observed between the two cell populations. These results show that the chondrocytes in an equal time proliferate significantly more if the culture surface is enlarged during culturing, compared with culturing them on an equal, but not enlarged culture surface.

1. A method for in vitro proliferation of cells (4), wherein the cells (4) are seeded on a culture surface (1) and, adhering to the culture surface, are cultured in a culture medium, wherein, during cell proliferation, the culture surface (1) is enlarged continuously or in steps, wherein the cells (4) remain in the culture medium before and during culture surface enlargement, and wherein the culture surface enlargement, just as with passaging, is adapted to the number of cells which is growing due to the cell proliferation.

2. The method according to claim 1, wherein, during the culture surface enlargement, the cells (4) are left adhering to the culture surface, and the culture surface (1) is enlarged by expansion.

3. The method according to claim 1, wherein, during the culture surface enlargement, the cells (4) are left adhering to the culture surface, and the culture surface is enlarged by inserting further culture surface regions between regions of the culture surface (1), to which the cells adhere.

4. The method according to claim 3, wherein the culture surface (1) consists of a multitude of particles to which the cells adhere, and the surface is enlarged by inserting further particles between the particles, to which the cells adhere.

5. The method according to claim 3, wherein the culture surface (1) is the inner surface of a compressed, open-pored body (22), and the culture surface is enlarged by reducing a compression of the body (22), so that further pores are opened and new surface portions made available.

6. The method according to claim 1, wherein, before or during the culture surface enlargement, at least a part of the cells (4) are detached from the culture surface and are brought into suspension, and the culture surface is enlarged by adding at least one further culture surface region (1″).

7. The method according to claim 6, wherein the cells (4) are detached by shear forces, wherein the shear forces are dimensioned such that only cells in a division phase are detached.

8. The method according to claim 7, wherein the shear forces are produced by culture medium currents.

9. The method according to claim 8, wherein the cells (4) are detached mechanically from the culture surface (1, 1″).

10. A device for in vitro proliferation of cells (4) in a culture medium, wherein the cells adhere to a culture surface, said device comprising the culture surface (1) being flooded over or around by the culture medium, and means for renewing the culture medium flooding over or around the culture surface, wherein the device further comprises means for continuous or stepwise enlargement of the culture surface (1) being flooded over or around by the culture medium, said means being controlled for a culture surface enlargement that is adapted, just as with passaging, to the number of cells which is growing due to cell proliferation.

11. The device according to claim 10, wherein the culture surface (1) is one side of an expandable membrane (6), and the device comprises means for expanding the membrane (6).

12. The device according to claim 11, wherein the means for expanding the membrane (6) comprise a space (5) adjacent to a membrane side opposite the culture surface (1) and being filled with a fluid or a gas, in which space (5) means (7) for changing the pressure are provided.

13. The device according to claim 10, wherein the culture surface (1) is formed by a multitude of particles, and the device further comprises means for inserting further particles between the particles of the culture surface (1).

14. The device according to claim 13, wherein the means for inserting comprise a container (12) having a cross section that increases in an upward direction and a means (15) for displacing the particles in said container (12) in the upward direction.

15. The device according to claim 10, wherein the device comprises culture surface regions (1″) that are selectively flooded by the culture medium, wherein, for culture surface enlargement, additional ones of the culture surface regions (1″) are flooded, and wherein the device further comprises means for detaching at least part of the cells from the culture surface (1, 1″) and for suspending the detached cells in the culture medium.

16. The device according to claim 15, wherein the means for detaching are means for producing shear forces.

17. The device according to claim 15, wherein the means for detaching are conduits (40) that comprise permeable walls and outer surfaces being equipped as culture surfaces (1, 1″), wherein the device further comprises means for flowing an enzyme solution through the conduits, to be brought through the permeable walls into contact with cells adhering to the outer surfaces of the conduits.

18. The device according to claim 15, wherein the means for releasing are blades (50), brushes or scrapers that are capable of being moved along the culture surface (1, 1″).
